(19) United States
(12) Patent Application Publication
(10) Pub. No.: US 2026/0347952 A1
(43) Pub. Date: Nov. 5, 2026

(54) METHOD FOR SELECTING HIGH-EFFICIENCY MOLECULAR MARKER FOR ENHANCING SELECTION EFFICIENCY FOR TARGET TRAIT AND USAGE THEREOF

(71) Applicant: SEEDERS, Daejeon (KR)

(72) Inventor: Sung-Hwan JO, Daejeon (KR)

(73) Assignee: SEEDERS, Daejeon (KR)

(21) Appl. No.: 19/487,273

(22) PCT Filed: Nov. 13, 2024
(86) PCT No.: PCT/KR2024/096491

§ 371 Date: Nov. 24, 2025

(30) Foreign Application Priority Data
Nov. 13, 2023 (KR) 10-2023-0156028
Mar. 11, 2024 (KR) 10-2024-0033607

(51) Int. Cl. C12Q 1/6895

(52) U.S. Cl. CPC C12Q 1/6895; C12Q 2600/13; C12Q 2600/156

(57) ABSTRACT

Conventional molecular marker discovery techniques, such as QTL mapping or GWAS, have been used to search for molecular markers associated with a target trait. However, these conventional techniques have a limitation in that they cannot process hundreds of thousands or more of SNPs recently obtained through NGS, and the process of selecting a molecular marker with high actual selection efficiency from among candidate molecular markers is complex and time-consuming. By using the algorithm of the present disclosure, the molecular marker development process may be significantly shortened, and a molecular marker showing the maximum selection efficiency for a target trait may be effectively selected quickly, so that it can be utilized in various fields such as trait research, genetic research, or crop breeding.

### FIELD OF TECHNOLOGY

The present disclosure relates to a method for selecting a high-efficiency molecular marker to enhance selection efficiency for a target trait and a use thereof.

### BACKGROUND

A molecular marker is an analytical tool that detects various mutations within a genome and is utilized in various fields such as biology, genetic research, and trait analysis. By utilizing the molecular markers, the relationship between genes and traits may be studied, and individuals with a desired target trait may be selected early without the morphological selection process. As the scope of application of the molecular markers expands, the rapid and accurate acquisition of trait-related genes or molecular markers has become a key tool in the field of crop breeding, and is also essential for improving the selection efficiency of quantitative traits, such as productivity, quality, disease resistance, and adaptability in crops. However, the search for molecular markers with high individual selection efficiency is still very limited.

Heretofore, conventional molecular marker search technologies such as QTL mapping and Genome-Wide Association Study (GWAS) have been mainly used to search for trait-associated molecular markers. QTL mapping is a method of finding a chromosomal location or a molecular marker associated with a trait using a crossbred population, and representative methods for selecting markers associated with traits include the JoinMap and Windows QTL Cartographer programs. These programs are limited in that the acceptable number of molecular markers is restricted to a few thousand, making them incapable of processing the hundreds of thousands of SNPs recently acquired through next-generation sequencing (NGS). GWAS is a method of finding molecular markers with a high correlation with traits through statistical analysis, and is one of the most widely used techniques for searching genetic associations without limitations on the number of molecular markers. However, this method is complex and time-consuming in the process of selecting molecular markers with high selection efficiency among candidate molecular markers.

Because the effective selection and utilization of molecular markers is a critical factor in crop breeding and individual selection processes, the development of detectable molecular markers for a greater number of traits is increasing. Structural mutations in genes occurring within the genome of an organism may now be investigated and secured in large quantities across the entire genome in a short period of time due to the development of a large-scale base sequence analysis device called NGS. However, as the number of discovered structural mutations has reached hundreds of thousands to millions, the complexity associated with molecular marker selection has significantly increased. Accordingly, it is necessary to develop a molecular marker selection algorithm capable of obtaining maximum selection efficiency for a target trait using a minimum number of molecular markers from large-scale structural mutations (for example, SNPs, Indels) existing across the entire genome identified through NGS or the like.

Meanwhile, Korean Patent Laid-Open Publication No. 2603207 discloses a ‘method for discovering SNP markers associated with phenotypes using statistical regularization method and selection probability’, but does not describe the ‘method for selecting a high-efficiency molecular marker to enhance selection efficiency for a target trait and the use thereof’ of the present disclosure, which sorts SNPs according to trait values within a population and assigns different weights to the genotypes of individuals distributed at both ends of the trait values and the genotypes of individuals distributed in the middle to select SNP markers highly associated with traits at both ends.

### SUMMARY OF THE INVENTION

### Technical Problem

The present disclosure was derived from the above-mentioned needs, and the inventors of the present disclosure have developed a new algorithm for selecting SNP markers with the highest selection efficiency for target traits compared to previously used QTL (Quantitative trait locus) mapping or GWAS. Specifically, NGS technology was used to obtain genome sequence data from F₂ crossbred populations or collected genetic resources, and large-scale SNPs existing within the population were searched. Next, the inventors developed an algorithm that may select SNP markers with high heritability and excellent selection efficiency for individuals related to the target trait by measuring the trait value for the target trait and assigning weights to SNPs according to the measured trait value. Thereafter, experimental verification was performed using an SNP-based KASP primer set selected through the above algorithm. In conclusion, the present disclosure was completed by confirming that when SNP markers selected through the new algorithm of the present disclosure are used rather than SNP markers selected through conventional QTL mapping or GWAS, individuals for the target trait may be selected more effectively, and that among numerous SNP markers searched through QTL mapping or GWAS, SNP markers closely linked to the target trait and with improved selection efficiency may be selected.

### Technical Solution

An embodiment of the present disclosure provides a method for determining, detecting, selecting, or isolating a single nucleotide polymorphism (SNP) marker associated with a target trait, including:
(1) a step of calculating a score (Sj) for each locus using [Formula 1] by assigning different weights according to variance based on a genotype of an individual distributed at both ends of aligned trait values and a genotype of an individual having a median value of a target trait, for each of at least ten or more reference SNPs; and

$${S}_{j} = {\sum }_{i = 1}^{N} {( {X}_{i} - \overline{X} )}^{2} \cdot {G}_{ij} \qquad [ Formula 1 ]$$

(In [Formula 1], N denotes the total number of samples, Xᵢ denotes the trait value of the i-th sample, X̅ denotes the average of the trait values of all samples, and Gᵢⱼ denotes the sample-specific weighting factor (where Gᵢⱼ=1 if the sample is a homozygous type and the SNP base matches the target trait, Gᵢⱼ=−1 if the sample is a homozygous type and the SNP base does not match the target trait, and Gᵢⱼ=0 if the sample base information is unavailable or the sample is a heterozygous type).)
(2) a step of selecting top Q loci among markers (positions) between 1 and L using [Formula 2] for the score (Sj) of each locus in the step (1).

TopQPositions_{common}∩_{p∈{5, 10, 20}}SelectTopQ({S_{j, p}|j=1, . . . ,L}) [Formula 2]

(In [Formula 2], L denotes the last marker (position), P denotes the percentage of samples selected from both ends (upper and lower) in step (1), and Q denotes an integer from 1 to 100.)

Another embodiment of the present disclosure provides a method for producing a plant or seed population, including: a step of selecting a plant or seed population according to the method for determining, detecting, selecting, or isolating the target-trait-associated SNP marker; and crossing or self-fertilizing the selected one or more plants, or plants grown from the selected seeds.

Another embodiment of the present disclosure provides a method for introducing or increasing expression of a target trait-related SNP in a subject, including: a step of determining, detecting, selecting, or isolating a target trait-related SNP marker according to the method for determining, detecting, selecting, or isolating the target-trait-associated SNP marker; and a step of expressing or increasing the expression of the target trait-related SNP in the subject.

Another embodiment of the present disclosure provides a method for producing a plant having a target trait, including: a step of determining, detecting, selecting, or isolating a target trait-related SNP marker according to the method for determining, detecting, selecting, or isolating the target-trait-associated SNP marker; and a step of expressing or increasing the expression of the target trait-related SNP in the subject.

In addition, the present disclosure provides a plant having a target trait, which is produced by a method of introducing or increasing the expression of an SNP related to the target trait in the subject, or a method of producing a plant having the target trait.

### Advantageous Effects

Conventional molecular marker discovery techniques, such as QTL mapping or GWAS, have been used to search for molecular markers associated with a target trait. However, these conventional techniques have a limitation in that they cannot process hundreds of thousands or more of SNPs recently obtained through next-generation sequencing (NGS), and the process of selecting a molecular marker with high actual selection efficiency from among candidate molecular markers is complex and time-consuming. By using the algorithm of the present disclosure, the molecular marker development process may be significantly shortened, and a molecular marker showing the maximum selection efficiency for a target trait may be effectively selected quickly, so that it can be utilized in various fields such as trait research, genetic research, or crop breeding.

### BRIEF DESCRIPTION OF THE DRAWINGS

FIG. 1 shows a result of QTL mapping for a trait related to pepper fruit ripening.

Red arrow: SNP with the highest LOD (Logarithm of the odds) on chromosome 10.

FIG. 2 shows a result of performing a GWAS using the Genome Association and Prediction Integrated Tool (GAPIT) program for traits related to pepper fruit ripening.

Red boxes: SNPs with high LOD on chromosomes 10 and 6.

FIG. 3 shows a result of KASP analysis performed on parental lines, their F2 populations, and fast-ripening individuals corresponding to the comparison group using a primer set for KASP based on the candidate SNP marker ‘C10674K’ on chromosome 10 associated with ripening traits of pepper fruits.

FIG. 4 shows a result of KASP analysis performed on parental lines, their F2 populations, and fast-ripening individuals corresponding to the comparison group using a primer set for KASP based on the candidate SNP marker ‘C06233B’ on chromosome 6 associated with ripening traits of pepper fruits.

FIG. 5 shows a distribution view of SNP marker selection values associated with pepper fruit ripeness analyzed using the algorithm of the present disclosure.

Orange indicates SNP markers highly correlated with pepper fruit coloring traits selected using the algorithm of the present disclosure, while green indicates the region with the greatest variation among SNP markers passing through the best fit line.

FIG. 6 shows a result of KASP analysis performed on parental lines, their F2 populations, and fast-ripening individuals corresponding to the comparison group using a primer set for KASP based on the SNP marker ‘C09222M’ associated with ripeness of pepper fruits selected through the algorithm of the present disclosure.

FIG. 7 shows a result of QTL mapping for a trait related to resistance to radish Fusarium wilt.

Red arrow: SNP with the highest LOD on chromosome 7.

FIG. 8 shows a result of performing GWAS using the GAPIT and TASEEL (Trait Analysis by aSSociation) programs for a trait related to resistance to radish Fusarium wilt.

Red box: SNP with the highest LOD on chromosome 7.

FIG. 9 shows a distribution view of SNP marker selection values associated with resistance to radish Fusarium wilt analyzed using the algorithm of the present disclosure.

FIG. 10 shows a chromosomal distribution diagram of SNP markers associated with resistance to radish Fusarium wilt selected using the algorithm of the present disclosure.

FIG. 11 shows a result of performing KASP analysis on parental lines (female parent, resistance(S); male parent, sensitivity (R)) and recombinant inbred lines (RILs), using a KASP primer set based on the radish Fusarium wilt resistance-associated SNP marker ‘R0712812M’ selected through the algorithm of the present disclosure.

FIG. 12 shows a PCR result confirming the presence or absence of Pun1, a spicy taste-related gene, in the red pepper genetic resource.

Lane 1: C25, Lane 2: C26, Lane 3: C27, Lane 4: C28, Lane 5: C29.

FIG. 13 shows a result of performing GWAS using the GAPIT program on traits related to the spiciness of red pepper.

Red boxes: SNPs with high LOD on chromosomes 2, 9, and 10.

FIG. 14 shows a distribution view of SNP marker selection values associated with the spiciness of peppers analyzed using the algorithm of the present disclosure.

FIG. 15 shows a chromosomal distribution view of SNP markers associated with pepper spiciness selected using the algorithm of the present disclosure.

### DETAILED DESCRIPTION OF THE EMBODIMENTS

To achieve an objective of the present disclosure, the present disclosure provides a method for determining, detecting, selecting, or isolating a single nucleotide polymorphism (SNP) marker associated with a target trait, including:
(1) a step of calculating a score (Sj) for each locus using [Formula 1] by assigning different weights according to variance based on a genotype of an individual distributed at both ends of aligned trait values and a genotype of an individual having a median value of a target trait, for each of at least ten or more reference SNPs; and

$${S}_{j} = {\sum }_{i = 1}^{N} {( {X}_{i} - \overline{X} )}^{2} \cdot {G}_{ij} \qquad [ Formula 1 ]$$

(In [Formula 1], N denotes the total number of samples, Xᵢ denotes the trait value of the i-th sample, X̅ denotes the average of the trait values of all samples, and Gᵢⱼ denotes the sample-specific weighting factor (where Gᵢⱼ=1 if the sample is a homozygous type and the SNP base matches the target trait, Gᵢⱼ=−1 if the sample is a homozygous type and the SNP base does not match the target trait, and Gᵢⱼ=0 if the sample base information is unavailable or the sample is a heterozygous type).)
(2) a step of selecting top Q loci among markers (positions) between 1 and L using [Formula 2] for the score (Sj) of each locus in the step (1).

TopQPositions_{common}=∩_{p∈{5, 10, 20}}Select TopQ({S_{j, p}|j=1, . . . ,L}) [Formula 2]

(In [Formula 2], L denotes the last marker (position), P denotes the percentage of samples selected from both ends (upper and lower) in step (1), and Q denotes an integer from 1 to 100.)

In the method according to the embodiment of the present disclosure, the Q may be an integer from 1 to 100, and preferably may be an integer from 1 to 10, but is not limited thereto.

In the method according to the embodiment of the present disclosure, the method may additionally include a step of obtaining a reference SNP by analyzing genome sequence information of each individual or progeny population obtained by crossing paternal and maternal lines.

In the method according to the embodiment of the present disclosure, the genotype of the individual may be obtained by collecting SNPs through analysis of genomic sequence information of each individual or progeny population obtained by crossing the paternal line and maternal line.

In the method according to the embodiment of the present disclosure, the method may additionally include a step of measuring a trait value for the target trait for each individual or progeny population obtained by crossing the paternal line and maternal line, and listing SNPs in order of the trait values, but is not limited thereto.

In the method according to the embodiment of the present disclosure, the trait values are measured by target traits for each individual or progeny population obtained by crossing the paternal and maternal lines, and the SNPs may be listed in order of trait values.

In the method according to the embodiment of the present disclosure, the SNP in step (1) may be selected as an SNP with a minor allele frequency (MAF) of 5% or more, a missing data of 30% or less, and a homozygous SNP among polymorphic SNPs between parental lines, but is not limited thereto.

In the method according to the embodiment of the present disclosure, the target trait-associated SNP marker may be selected by repeatedly performing steps (1) and (2) on individuals at both ends obtained by sequentially applying the ranges of significance probability (p-value) of 5% or less, 10% or less, and 15% or less to both ends of the aligned trait values of step (1), but is not limited thereto.

In the method according to an embodiment of the present disclosure, the target trait-associated SNP marker may be selected from a subject. The subject may include one or more selected from the group consisting of a seed, a plant, an animal, a bacterium, and an insect, and preferably may include a plant or a seed, but is not limited thereto.

Additionally, in the method according to an embodiment of the present disclosure, the target trait-associated SNP marker may be selected from a sample. The sample may include one or more selected from the group consisting of a seed, a plant, an animal, a bacterium, and an insect, and preferably may include a plant or a seed, but is not limited thereto.

In the method according to the embodiment of the present disclosure, the target trait may have a characteristic including ripening of pepper fruit, a characteristic including resistance to radish Fusarium wilt, or a characteristic including the spicy gene (Pun1) of pepper, but is not limited thereto. In addition, the target trait may have a characteristic including a resistance gene R1, R8, or Rpi-amr1 against Phytophthora pathogens, but is not limited thereto. In addition, the target trait may have a characteristic including one or more selected from the group consisting of herbicide resistance, disease resistance, insect or pest resistance, fungal disease resistance, virus resistance, nematode resistance, bacterial disease resistance, fatty acid biosynthesis, starch biosynthesis, increased grain yield, increased oil content, nutritional improvement, increased growth rate, fruit ripening, increased yield, improved stress tolerance, improved environmental or chemical tolerance, change in morphological characteristics, improved digestibility, industrial enzyme production, improved flavor, nitrogen fixation, and a combination thereof, but is not limited thereto.

In the method according to an embodiment of the present disclosure, the method may provide better target trait selection efficiency compared to a Genome-Wide Association Study (GWAS) program and/or QTL mapping.

In the method according to the embodiment of the present disclosure, the method may specifically include
(1) a step of obtaining a single nucleotide polymorphism (SNP) by analyzing genomic sequence information of each individual or progeny population obtained by crossing a male parent and a female parent;
(2) a step of measuring a trait value for a target trait in each individual or progeny population obtained by crossing the male parent and the female parent and listing the SNPs in the order of the trait values;
(3) a step of calculating a score (Sj) of each locus using the following [Formula 1] by applying different weights according to variance based on a genotype of an individual distributed at both ends of the aligned trait values and a genotype of an individual having a median value of the target trait; and

$${S}_{j} = {\sum }_{i = 1}^{N} {( {X}_{i} - \overline{X} )}^{2} \cdot {G}_{ij} \qquad [ Formula 1 ]$$

(In [Formula 1], N denotes the total number of samples, Xᵢ denotes the trait value of the i-th sample, X̅ denotes the average of the trait values of all samples, and Gᵢⱼ denotes the sample-specific weighting factor (where Gᵢⱼ=1 if the sample is a homozygous type and the SNP base matches the target trait, Gᵢⱼ=−1 if the sample is a homozygous type and the SNP base does not match the target trait, and Gᵢⱼ=0 if the sample base information is unavailable or the sample is a heterozygous type).)
(4) a step of selecting a top 10 loci among markers (positions) between 1 and L using [Formula 3] for the score (Sj) of each locus in step (3), but is not limited thereto.

Top10Positions_{common}=∩_{p∈{5, 10, 20}}SelectTop10({S_{j, p}|j=1, . . . ,L}) [Formula 3]

(In [Formula 2], L denotes the last marker (position), and P denotes the percentage of samples selected from both ends (upper and lower) in step (1))

In the method according to the embodiment of the present disclosure, the sequence information analysis in step (1) may be performed through next-generation base sequencing (NGS), but is not limited thereto.

In the method according to the embodiment of the present disclosure, the target trait in step (2) may be an agronomic trait of a crop (for example, plant height, fruit color, ripening period, taste, and the like) or a trait such as disease resistance, but is not limited thereto.

In the method according to the embodiment of the present disclosure, the trait values in step (2) may be aligned in ascending or descending order, but are not limited thereto.

In the method according to the embodiment of the present disclosure, the SNP in the step (1) may be selected as an SNP with a minor allele frequency (MAF) of 5% or more, a missing data of 30% or less, and a homozygous SNP among polymorphic SNPs between parental lines, but is not limited thereto.

In addition, step (2) includes a process of aligning the SNPs of each individual according to the trait values, and the reason this process is necessary is that it is difficult to determine the association between the trait and the sample by only looking at the SNP results without the trait values. The measurement method of the trait value for the target trait varies depending on the type of target trait, and in the case of a crop, it may be performed according to the characteristic investigation standards of the Korea Seed & Variety Service (https://www.seed.go.kr/seed/192/subview.do), but is not particularly limited thereto.

In the method according to the embodiment of the present disclosure, both ends in step (3) mean individuals having characteristics opposite to each other for a specific trait. For example, when fruit ripeness is set as the target trait, the trait values of the progeny obtained by crossing the female and male parents are measured and then aligned in descending or ascending order, both ends include samples with early-ripening fruits and samples with late-ripening fruits, which have opposite characteristics. Accordingly, the present disclosure assigns different weights according to variance based on the genotype of the individual distributed at both ends and the genotype of the individual having the median value of the trait, so that a high value is assigned to SNPs that may effectively select the individuals at both ends.

The target trait-associated SNP marker selected through the method according to the embodiment of the present disclosure is selected by repeatedly performing steps (3) and (4) on individuals at both ends obtained by sequentially applying the ranges of significance probability (p-value) of 5% or less, 10% or less, and 15% or less to both ends of the aligned trait values of step (3).

Specifically, when conducting genetic analysis, more than 96 samples are used for a specific population to ensure that various groups are evenly represented in the entire population, and most analyses are performed in multiples of 96 samples (192, 288, . . . ).

Accordingly, in the present disclosure, the first calculation was performed with 5% as the standard for statistical significance in 96 samples (approximately 100 samples) for analyzing the population, and this is because the larger the number of samples (the larger the population), the more it follows a normal distribution, so the calculation was performed based on samples having trait values with a significance probability (p-value) of 5% or less. However, if the process is performed only based on a p-value 0.05 (5%) as described above, samples distributed in the middle (samples having intermediate trait values) are not considered, so the results will be biased towards the samples at both ends, and the curse of dimensionality (a phenomenon in which it becomes impossible to make a determination due to numerous candidate markers when selecting a marker that can explain a single trait with a small number of samples) may occur.

For this reason, a different weight is assigned to the genotypes of individuals having trait values with a p-value of 5% or less and the genotypes of individuals distributed in the middle, at the both ends of the sorted trait values in step (3), and the SNP is selected using [Formula 1] and [Formula 3] of step (4), and a different weight is assigned to the genotypes of individuals having trait values with a p-value of 10% or less and the genotypes of individuals distributed in the middle, at the both ends of the sorted trait values in step (3), and the SNP is selected through the same process as above, and further, a different weight is assigned to the genotypes of individuals having trait values with a p-value of 15% or less and the genotypes of individuals distributed in the middle, at the both ends of the sorted trait values in step (3), and the SNP is selected through the same process as above. When steps (3) and (4) are repeatedly performed by applying different p-values to the both ends of the trait values as described above, if SNPs in the same LD (Linkage Disequilibrium) block are repeatedly selected, it is determined that there is a high probability that the SNPs selected through this process are associated with the actual trait value.

LD is analyzed by calculating recombination using SNP markers that move together with a gene, then identifying the associated information, and predicting the relative distance between genes. LD blocks are regions of a specific genome that are so short or densely packed with genes that crossover rarely occurs over generations, and therefore, the genetic information in these regions is almost identical and is preserved over generations.

The present disclosure also provides a method for producing a plant or seed population, including: a step of selecting a population of plants or seeds according to a method for determining, detecting, selecting, or isolating the target-trait-associated SNP marker; and
crossing or self-fertilizing the selected one or more plants, or plants grown from the selected seeds.

In the method for producing the plant or seed population according to the embodiment of the present disclosure, the selected population may have a target trait of improved ripening of pepper fruits, a target trait of improved resistance to radish Fusarium wilt, or a target trait having improved pungency gene (Pun1) of pepper, but is not limited thereto. In addition, the selected population may have a target trait having an enhanced resistance gene R1, R8 or Rpi-amr1 against Phytophthora pathogen, but is not limited thereto.

The present disclosure provides a method for introducing or increasing expression of a target trait-related SNP in a subject, comprising:
a step of determining, detecting, selecting or isolating the target trait-related SNP marker according to the method of determining, detecting, selecting or isolating the target trait-related SNP marker, and
   a step of expressing or increasing the expression of the target trait-related SNP in the subject.

The present disclosure provides a method for producing a plant having a target trait, comprising:
a step of determining, detecting, selecting or isolating the target trait-related SNP marker according to the method of determining, detecting, selecting or isolating the target trait-related SNP marker, and
a step of expressing or increasing the expression of the target trait-related SNP in the subject.

In the method for introducing or increasing the expression of the target trait-related SNP in the subject or the method for producing the plant having the target trait according to the embodiment of the present disclosure, the subject is a plant, and the method may further include crossing the plant with another plant of the same species to produce a progeny plant having the target trait, but is not limited thereto.

The present disclosure also provides a plant having a target trait produced by a method of introducing or increasing the expression of an SNP related to the target trait in the subject, or a method of producing a plant having a target trait according to an embodiment of the present disclosure.

Hereinafter, the present disclosure will be described in detail with reference to examples. However, the following examples are merely illustrative of the present disclosure, and the scope of the present disclosure is not limited thereto.

### Example 1. Selection of Markers Related to Ripening of Pepper Fruits

As a first case, a study on ripening-related traits of pepper fruits was performed, and a molecular marker related to coloring was developed using an F₂ population of peppers. In this case, the present disclosure discovered a highly efficient molecular marker corresponding to a Type II error, excluding markers corresponding to a Type I error commonly searched through QTL mapping or GWAS.

### 1-1. Analysis Material

Using parental lines that exhibited differences in fruit ripening time, F₂ populations were bred, and the ripening time of both parental lines and F₂ populations was measured. Young leaves of each sample were collected, and genomic DNA was extracted and used as a GBS (Genotyping-by-sequencing) sample.

### 1-2. Biological Information Analysis and SNP Statistical Analysis

Genomic DNA was extracted from young leaves of each sample for a total of 98 lines including the parent lines, digested with the restriction enzyme ApeKI, and a GBS library was prepared, and the base sequence was produced using Illumina's Hiseq X10. Using the produced base sequence, SNPs were selected through the bioinformatics pipeline within Seeders. SNPs satisfying the conditions of MAF (minor allele frequency) of 5% or more and missing data of 30% or less were selected, and the chromosomal location within the reference genome (https://solgenomics.net/organism/Capsicum_annuum/genome) the and presence of polymorphic SNPs between parents were checked, and additional filtering processes were performed as necessary to use them in the next analysis.

TABLE 1
Summary of SNP filter process

| Filter | | SNP matrix |
|---|---|---|
| step | Filter item | loci |
| 1 | Total SNP matrix | 12,550,758 |
| 2 | MAF (minor allele frequency) >5% *¹ | 4,376,351 |
| 3 | MAF >20%, locus at chromosome level | 4,153,706 |
| 3 | Missing data <30% *² | 116,729 |
| 4 | MAF >5%, Missing data <30% | 14,057 |
| 5 | Parental polymorphic SNP homo-type locus *³ | 9,981 |
| 6 | Chromosome level locus | 9,698 |
| 7 | MAF >20%, Missing data <30%, Parental | 8,367 |
| | polymorphic SNP homo-type locus | |
| 8 | SNP selection for linkage map formation *⁴ | 2,250 |

*¹ SNPs with a MAF greater than 5% are selected from the entire sample at the corresponding locus.
*² SNPs with less than 30% missing data are selected from the entire sample at the corresponding locus.
*³ Homozygous-type SNPs are selected among the polymorphic SNPs between the two parental samples.
*⁴ SNPs are selected based on the maximum locus limit of the JoinMap program input.

### 1-3. Selection of Coloring-Related Molecular Markers Through QTL Mapping

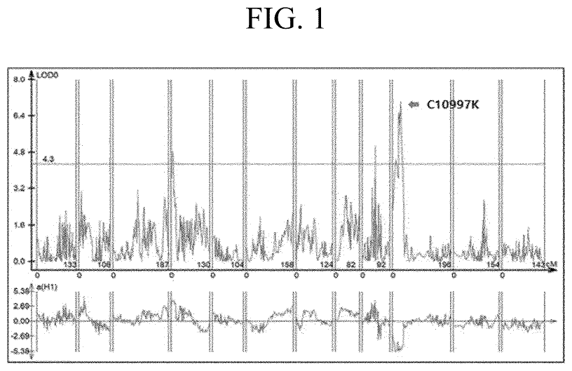

As a result of using the JoinMap program for the 2,250 SNP loci finally selected, 2,242 SNP loci were used to form a linkage map including 12 linkage groups. Through the Windows QTL Cartographer program, the QTL loci showing the highest LOD (Logarithm of the odds) in linkage group 10 were identified (FIG. 1), and in addition, QTL loci showing a standard LOD value of 4.3 or higher were detected on chromosomes 4 and 9.

### 1-4. Selection of Molecular Markers Through GWAS Using GAPIT (Genome Association and Prediction Integrated Tool) Program

Due to the limitations of programs developed to date, the number of SNP markers actually used for linkage map formation is limited to 2,000 to 3,000, but GWAS offers the advantage of performing linkage analysis with a sufficient number of SNPs, overcoming the numerical limitations of markers. In the present disclosure, a linkage analysis on the ripeness of pepper fruits was conducted using GAPIT (applying 8 models), the most commonly used GWAS program, using 8,367 refined SNP loci.

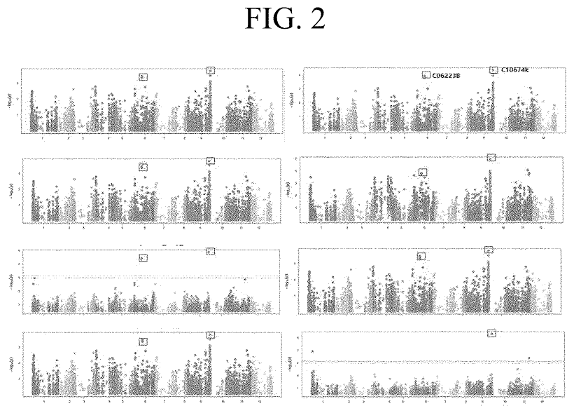

As a result, the most significant signal was confirmed at the C10674K marker on Chromosome 10 in all GWAS models, followed by a signal confirmed at the C06233B marker located on Chromosome 6. Accordingly, the C10674K and C06233B markers were selected as candidate markers (FIG. 2).

### 1-5. Candidate Molecule Marker Screening Using Primer Sets for KASP

In order to measure the selection effect of individuals using the candidate SNP markers selected through the QTL mapping and GWAS using GAPIT, a primer set for KASP based on the candidate SNPs was prepared, and the selection efficiency was confirmed on individuals for which trait information was already known.

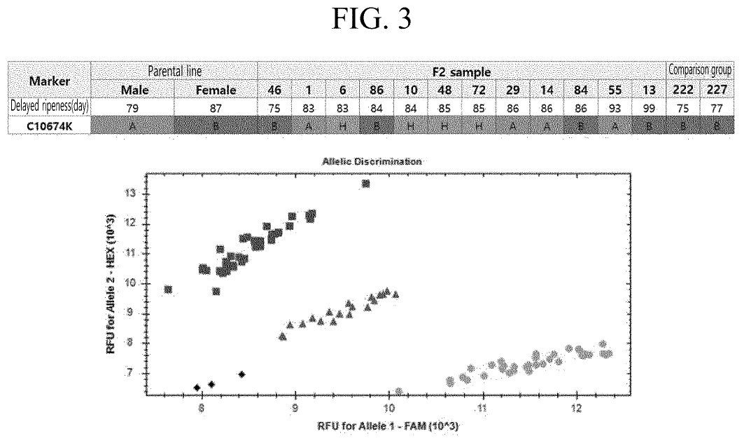

First, using the primer set for KASP based on the C10674K marker selected frp, chromosome 10, which commonly showed the highest signal through QTL mapping and GWAS, KASP analysis was performed on parental lines with different fruit ripening times, their F₂ populations, and fast-ripening individuals (control group), and as a result, the genotype of the late-ripening female parent was B, and the genotype of the relatively early-ripening male parent was A (FIG. 3), but the comparison group (early-ripening individuals) showed genotype B, which did not match the genotype of the male parent.

Additionally, in the F2 population of FIG. 3, the median value of the trait is 86, so if the trait value is less than 86, it must have base A, and if it is 86 or more, it must have base B. However, as shown in FIG. 3, among the F₂ samples with a trait value of less than 86, only 1 out of 7 samples had base A, and among the F₂ samples with a trait value of 86 or more, only 2 out of 5 samples had base B, showing a tendency to be unsuitable for use as a marker for the F₂ population. Through this, it was seen that the C10674K marker selected on chromosome 10 was not suitable for selecting pepper individuals with different ripening times.

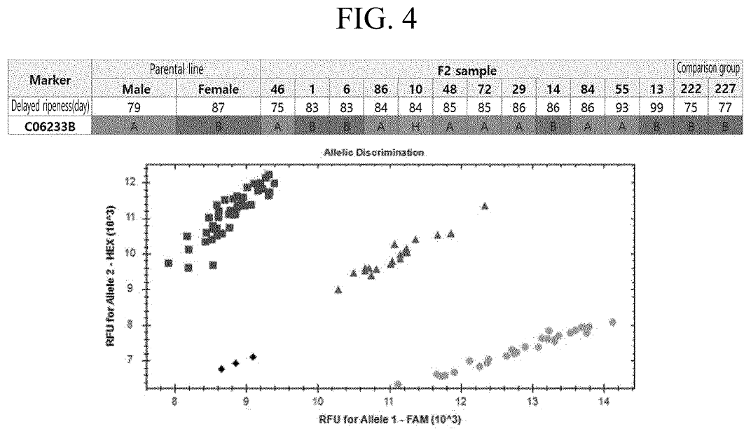

In addition, KASP analysis was performed on the same sample as above using a primer set for KASP based on the C06233B marker selected from chromosome 6 by GWAS, and as a result, as above, the genotype of the late-ripening female parent was B, and the genotype of the relatively early-ripening parent was A, but the comparison group (early-ripening individuals) showed genotype B, confirming that it did not match the genotype of the male parent (FIG. 4). Through this, it was seen that the C06233B marker was also not suitable for selecting pepper individuals with different ripening times.

### 1-6. Development of a Novel Algorithm for Selecting SNP Markers Related to Coloring of Pepper Fruit

Through the results above, it was seen that the selection efficiency of the SNP markers selected through QTL mapping and GWAS was poor, and a new algorithm was developed to secure markers with enhanced selection efficiency.

In the first step, sequence information of a progeny population obtained by crossing a male parent and a female parent or sequence information of collected resources was analyzed to secure SNPs.

In the second step, the trait values of each sample for the target trait were measured in the progeny population obtained by crossing the male parent and the female parent or in the collected resource, sorted in ascending or descending order, and then the SNPs of each individual were listed in the order of the trait values.

In the third step, by assigning different weights according to variance based on the genotypes of individuals distributed at both ends that show different trait characteristics in the sorted trait values, and the genotype of the individual having the median value of the trait, a higher value is assigned to the SNP that effectively selects the individuals at the both ends (Formula 1). That is, the score (Sj) for each locus was calculated by assigning a weight to the additive effect.

$${S}_{j} = {\sum }_{i = 1}^{N} {( {X}_{i} - \overline{X} )}^{2} \cdot {G}_{ij} \qquad [ Formula 1 ]$$

(In [Formula 1], N denotes the total number of samples, Xᵢ denotes the trait value of the i-th sample, X̅ denotes the average of the trait values of all samples, and Gᵢⱼ denotes the sample-specific weighting factor (where Gᵢⱼ=1 if the sample is a homozygous type and the SNP base matches the target trait, Gᵢⱼ=−1 if the sample is a homozygous type and the SNP base does not match the target trait, and Gᵢⱼ=0 if the sample base information is unavailable or the sample is a heterozygous type).)

In the fourth step, the top 10 loci among markers (positions) between 1 and L were selected for Sj (Formula 3).

Top10Positions_{common}=∩_{p∈{5, 10, 20}}SelectTop10({S_{j, p}|j=1, . . . ,L}) [Formula 3]

(In [Formula 2], L denotes the last marker (position), and P denotes the percentage of samples selected from both ends (upper and lower) in step (1))

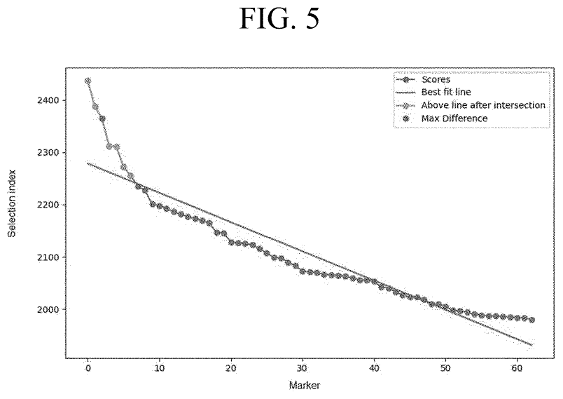

By applying the coloring-related traits of pepper fruits to the above-described algorithm, 10 markers belonging to the top group on the graph were selected and marked in orange, as shown in FIG. 5, and information on the top 5 markers among the 10 selected markers is summarized in Table 2 below.

TABLE 2
Information on the top 5 markers selected using
the algorithm of the present disclosure

| | Marker | | Selection | Best |
|---|---|---|---|---|
| Rank | Name | Chromosome | Index | Fit Line |
| 1 | C09222M | 9 | 2465 | Pass |
| 2 | C09238A | 9 | 2389 | Pass |
| 3 | C09238B | 9 | 2367 | Pass |
| 4 | C09216A | 9 | 2308 | Pass |
| 5 | C09216B | 9 | 2306 | Pass |

As shown in Table 2, the SNP markers selected through the algorithm of the present disclosure were confirmed as having the highest selection efficiency on chromosome 9, unlike those identified through conventional QTL mapping and GWAS. Molecular markers passing through a best fit line may be candidate markers usable as selected markers, and these were mainly concentrated on chromosome 9. Among these, the marker with the highest value was named C09222M. The clustering of the markers selected as the top group in an LD block on the chromosome demonstrated results highly consistent with existing theory.

According to the QTL mapping results, the highest signal was observed on chromosome 10, followed by a relatively strong signal on chromosome 9. Although signals were observed on the same chromosome, the physical position of the SNP in the genome showed a difference of several Mbp. However, the C10674K marker on chromosome 10 and the C06233B marker on chromosome 6, which were selected by QTL mapping or GWAS, showed very low values when analyzed through the algorithm of the present disclosure, and were thus excluded from the candidate group.

### 1-7. Verification of the C09222M Marker Selected Through the Algorithm of the Present Disclosure

Using a primer set for KASP based on the C09222M marker selected through the algorithm of the present disclosure, KASP analysis was performed on parental lines with different ripening times of pepper fruits, their F2 populations, and individuals with early ripening (comparison group).

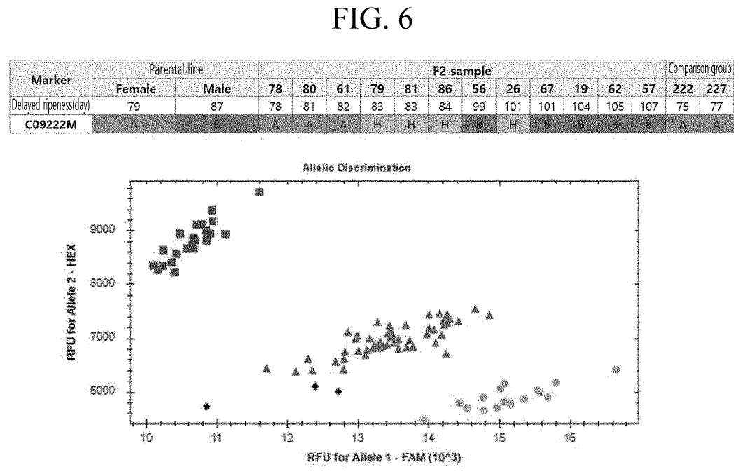

As a result, the genotype of the late-ripening female parent was B, and the genotype of the relatively early-ripening male parent was A, showing good polymorphism in the parental line, and the comparison group also showed genotype A, confirming that it was consistent with the genotype of the male parent (FIG. 6).

Additionally, in the F2 population of FIG. 6, the median value of the trait is 86, so if the trait value is less than 86, it must have base A, and if it is 86 or more, it must have base B. As shown in FIG. 6, among the F2 samples with a trait value of less than 86, only 3 out of 6 samples had base A, and among the F2 samples with a trait value of 86 or more, only 2 out of 6 samples had base B, indicating the suitability of using this marker for the F2 population.

Through this, it was seen that the C09222M marker selected through the algorithm of the present disclosure may effectively select pepper individuals with different ripening times.

### Example 2. Selection of Markers Related to Resistance to Radish Fusarium Wilt

In a second case, research was conducted on the trait related to resistance to radish Fusarium wilt, and a molecular marker linked to resistance to Fusarium wilt was developed using parent lines of radish (female parent, resistant; male parent, susceptible) and recombinant inbred lines (RILs).

### 2-1. Analysis Material

After breeding an RIL population using parent lines showing susceptibility or resistance to Fusarium wilt, 170 RIL lines were secured and subjected to disease inoculation tests and genotyping through GBS (Genotyping-by-sequencing). Young leaves of each sample were collected, and genomic DNA was extracted and used as a GBS (Genotyping-by-sequencing) sample.

### 2-2. Biological Information Analysis and SNP Statistical Analysis

Genomic DNA was extracted from young leaves of each sample from the parental lines (2 samples) and RIL 170 lines (170 samples), digested with the restriction enzyme ApeKI, and a GBS library was prepared, and the base sequence was produced using Illumina's Hiseq X10. Using the produced base sequence, SNPs were selected through the bioinformatics pipeline within Seeders. SNPs satisfying the conditions of MAF of 5% or more and missing data of 30% or less were selected, and the chromosomal location within the reference genome and the presence of polymorphic SNPs between parents were checked, and additional filtering processes were performed as necessary to use them in the next analysis.

TABLE 3
Summary of SNP filter process

| Filter step | Filter item | No. of SNPs |
|---|---|---|
| 1 | Total SNP | 724,590 |
| 2 | MAF (minor allele frequency) >5% *¹ | 225,124 |
| 3 | Missing data <30% *² | 171,019 |
| 4 | Missing data <30% & MAF >5% | 48,041 |
| 5 | SNP in chromosome *³ | 45,842 |
| 7 | Parent sample polymorphic homo- | 88,842 |
| | type locus *⁴ | |
| 8 | MAF (minor allele frequency) >20% | 69,337 |
| 9 | Missing data <10% | 20,526 |
| 10 | Missing data <10% & MAF >20% | 17,342 |
| 11 | Random selection | 1,576 |

*¹ SNPs with a MAF greater than 5% are selected from the entire sample at the corresponding locus.
*² SNPs with less than 30% missing data are selected from the entire sample at the corresponding locus.
*³ SNP loci secured at the chromosome level of the standard genome are selected.
*⁴ Homozygous-type SNPs are selected among the polymorphic SNPs between the two parental samples.

### 2-3. Selection of Molecular Markers Related to Resistance to Fusarium Wilt Through QTL Mapping

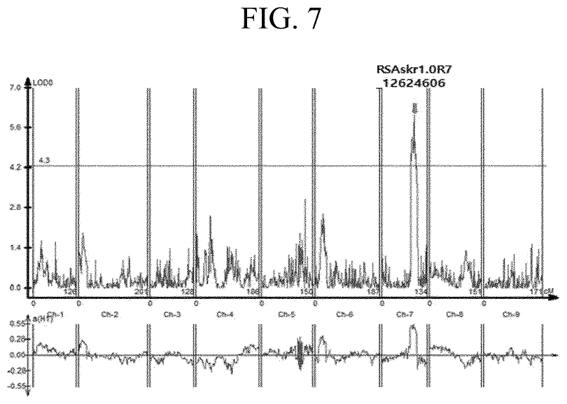

Finally, using the 1,576 ultimately selected SNP loci and the JoinMap program, 1,537 SNP loci were utilized to construct a linkage map including nine linkage groups. The QTL loci showing the highest LOD score in linkage group 7 were identified through the Windows QTL Cartographer program (FIG. 7).

2-4. Selection of Molecular Markers Through GWAS Using GAPIT and TASSEL (Trait Analysis by aSSociation) Programs

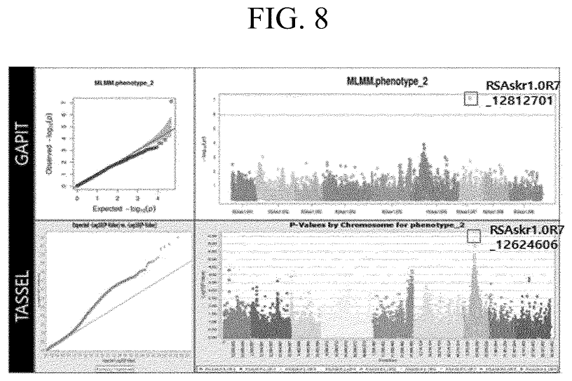

An association analysis for resistance to Fusarium wilt was performed using 45,842 SNP loci with GAPIT (applying the MLMM model) and TASSEL, which are the most commonly used GWAS programs. Both programs indicated the most significant signals on chromosome 7 (FIG. 8).

### 2-5. Selection of Markers Related to Resistance to Radish Fusarium Wilt Using the Algorithm of the Present Disclosure

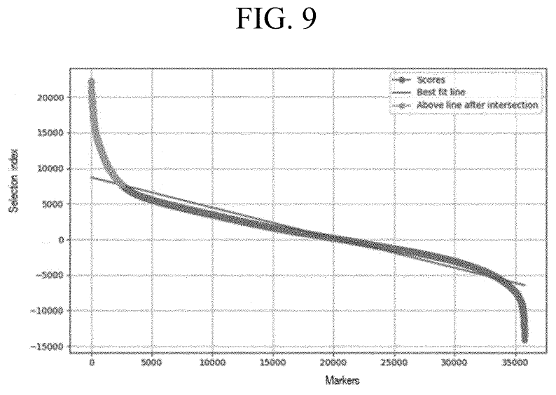

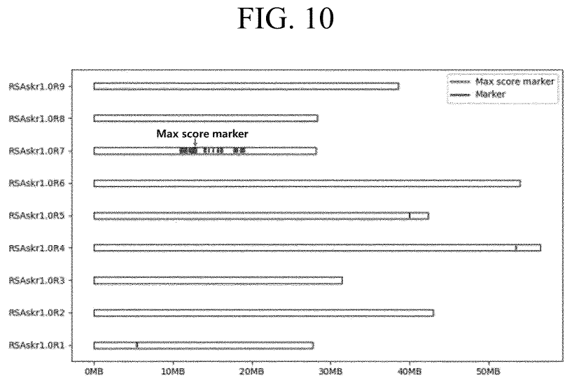

As a result of selecting SNP markers using the algorithm of the present disclosure, it was confirmed that the R0712812M marker with high selection efficiency exists on chromosome 7. Molecular markers passing through a best fit line may be candidate markers usable as selected markers (FIG. 9), and these were all concentrated on chromosome 7 (FIG. 10). Information on the top 5 markers among the 10 markers selected through the algorithm of the present disclosure is summarized in Table 4 below.

TABLE 4
Information on the top 5 markers selected
using the algorithm of the present disclosure

| | | | Selection | |
|---|---|---|---|---|
| Rank | Marker Name | Chromosome | Index | Best Fit Line |
| 1 | R0712812M | No. 7 | 23567 | Pass |
| 2 | R0712812A | 7 | 23561 | Pass |
| 3 | R0712812B | 7 | 23543 | Pass |
| 4 | R0712894A | 7 | 23538 | Pass |
| 5 | R0712812C | 7 | 23537 | Pass |

The marker candidates selected through the algorithm of the present disclosure were identical to the marker candidates selected through QTL mapping and GWAS, and showed results very consistent with existing theories as they were grouped in LD blocks. Accordingly, by using the algorithm of the present disclosure, it was possible to verify that the markers selected through QTL mapping or GWAS are markers with excellent selection efficiency.

### 2-6. Verification of the R0712812M Marker Selected Through the Algorithm of the Present Disclosure

KASP analysis was performed on the parental lines and an RIL population showing susceptibility or resistance to radish Fusarium wilt using a KASP primer set based on the R0712812M marker selected through the algorithm of the present disclosure.

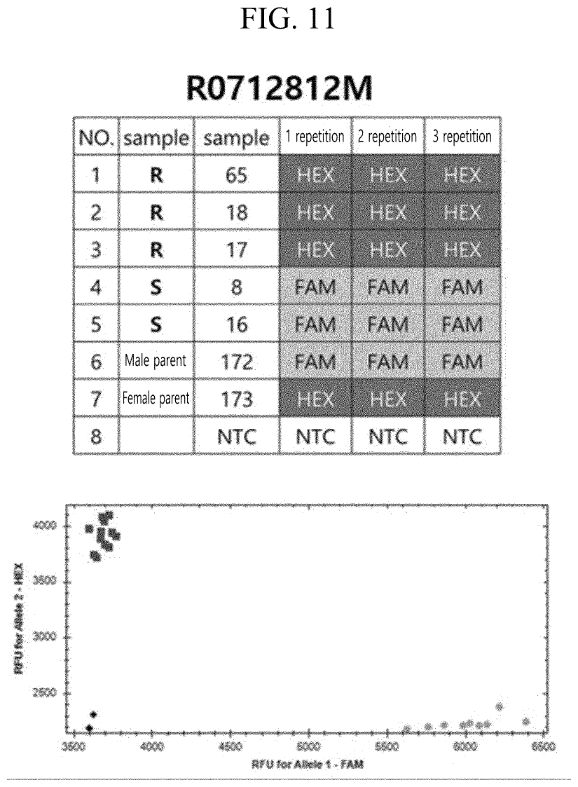

As a result, it was confirmed that the resistant maternal parent and two RIL lines were accurately distinguished from the susceptible paternal parent and two RIL lines (FIG. 11). Through this, it was seen that the R0712812M marker selected through the algorithm of the present disclosure may effectively select radish individuals having resistance to Fusarium wilt.

### Example 3. Selection of Markers Related to the Spiciness of Pepper

As a third case, it was verified whether the resequencing results of pepper genetic resource 111 could be utilized to accurately identify the genomic location of the Pun1 gene, which has previously been reported to be associated with pungency in peppers. The pepper genetic resource used this time is not an F2 population composed of progeny of specific parents as in Examples 1 and 2, but is an independently bred line with a more complex genetic composition. The applicability of the algorithm of the present disclosure was validated on this independent genetic population. The target trait was the known spicy taste-related gene, Pun1 (located on chromosome 2, an Indel marker), and the presence or absence of the gene was confirmed through PCR, and the PCR results were then recognized as a phenotype, and then, the algorithm of the present disclosure was applied to determine whether the selected molecular marker was appropriate.

### 3-1. Analysis Material

111 lines of typical pepper type and paprika type used as breeding lines were used as samples.

### 3-2. Biological Information Analysis and SNP Statistical Analysis

Genome sequences with 20× coverage per line were produced for the 111 lines of typical pepper type and paprika type using the Illumina platform (NovaSeq 6000). The produced genome sequences were preprocessed and mapped using the BWA program, and SNPs were selected using the Deepvariant program. Using the selected SNPs, an SNP matrix for all 111 lines was created, and the SNPs were refined based on MAF and missing data, thereby securing and utilizing a total of 11,242,804 SNPs (Table 5).

TABLE 5
Summary of SNP filter process

| Filter step | Filter item | SNP matrix loci |
|---|---|---|
| 1 | Total SNP matrix | 43,671,923 |
| 2 | MAF (minor allele frequency) >20% *¹ | 12,970,343 |
| 3 | Missing data <5% *² | 37,109,423 |
| 4 | MAF >5% and Missing data <30% | 11,242,804 |

*¹ SNPs with a MAF greater than 20% are selected from the entire sample at the corresponding locus.
*² SNPs with less than 5% missing data are selected from the entire sample at the corresponding locus.

### 3-3. Trait Investigation

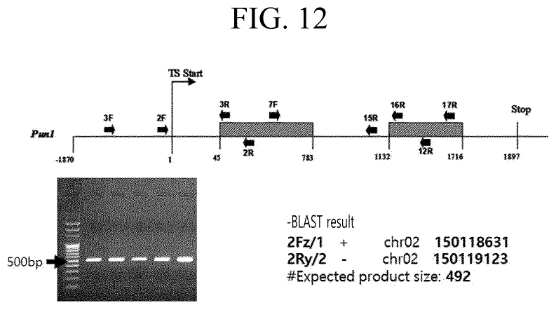

The presence of the spicy taste-related Pun1 gene was used as the target trait. To confirm the presence or absence of the Pun1 gene within 111 lines of chili pepper genetic resources, the PCR results using Pun1 gene-specific PCR primers disclosed in the paper by Hai Thi Hong Truong et al. (Horticulture Environment Biotechnology, 2009, 50 (4), 358-365) were summarized and used as the phenotype values. Using the reference genome sequence information (https://solgenomics.net/organism/Capsicum_annuum/genome), NCBI BLAST confirmed that it was located at 150 Mb on chromosome 2 (FIG. 12), which was used as a basis for determining the accuracy of future analyses.

### 3-4. Molecular Marker Selection Through GWAS Using the GAPIT Program

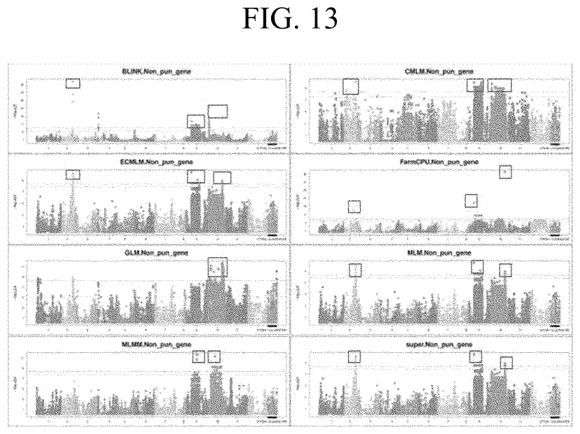

A linkage analysis on the spiciness of red pepper was conducted using GAPIT (applying 8 models), the most commonly used GWAS program, using 11,242,804 SNP loci. As a result, as shown in FIG. 13, most models showed signals as a significant marker group not only on chromosome 2 but also on chromosomes 9 and 10 in common, so additional experiments were performed.

The results showed considerable differences depending on the model used, and for example, the BLINK model showed the strongest signal on chromosome 2, while the FarmCPU model showed the strongest signal on chromosome 10. In most models, many signals were generated, making it difficult to distinguish between noise signals and true signals. In this way, in situations in which it is not known exactly where the gene is located, this phenomenon requires a lot of time and effort for additional verification experiments.

### 3-5. Selection of Markers Related to the Spiciness of Chili Peppers Using the Algorithm of the Present Disclosure

As a result of selecting SNP markers using the algorithm of the present disclosure, the strongest signal was observed on chromosome 2 among the several chromosomes identified through GWAS. The highest value was observed in the 150 Mb region of chromosome 2, consistent with the results confirmed by gel electrophoresis using the Universal Pun1 primer. Following chromosome 2, chromosome 10 showed the next highest value.

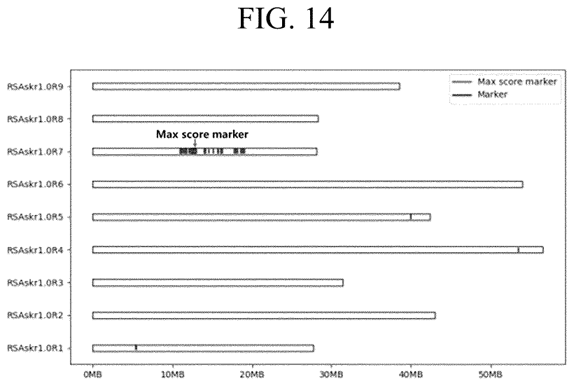

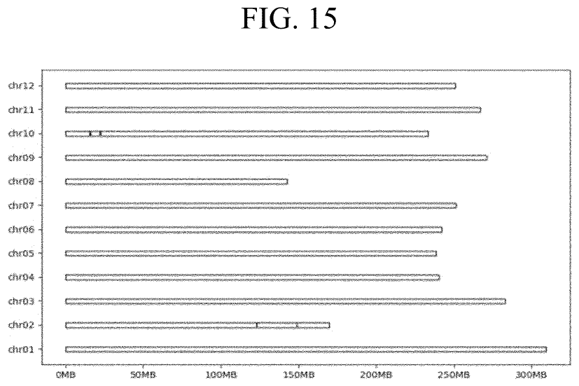

Molecular markers passing through a best fit line may be candidate markers usable as selected markers (FIG. 14), and these were mainly concentrated on chromosome 2 (FIG. 15). The clustering of the selected markers within the LD block indicated results that were highly consistent with existing theory, and it was confirmed that by utilizing the algorithm of the present disclosure, it is possible to select markers with a better selection efficiency among the markers selected through GWAS.

Information on the top 5 markers among the 10 markers selected through the algorithm of the present disclosure is summarized in Table 6 below.

TABLE 6
Information on the top 5 markers selected
using the algorithm of the present disclosure

| | Marker | | Selection | |
|---|---|---|---|---|
| Rank | Name | Chromosome | Index | best fit line |
| 1 | C02200A | 2 | 11.14 | Pass |
| 2 | C02200B | 2 | 11.09 | Pass |
| 3 | C02200C | 2 | 11.09 | Pass |
| 4 | C02200D | 2 | 11.07 | Pass |
| 5 | C02200E | 2 | 11.07 | Pass |

The examples of Examples 1 to 3 illustrate that the algorithm of the present disclosure provides excellent efficiency in the process of selecting markers associated with various traits, and may successfully contribute to the discovery and utilization of excellent candidate markers compared to conventional methods.

1. (canceled)

2. The method according to claim 34, wherein
the Q is an integer from 1 to 10.

3. The method according to claim 34, further comprising
a step of obtaining a reference SNP by analyzing genomic sequence information of each individual or progeny population obtained by crossing a paternal line and a maternal line.

4. The method according to claim 34, wherein
a genotype of the individual is obtained by collecting SNPs through analysis of genomic sequence information of each individual or progeny population obtained by crossing a paternal line and a maternal line.

5. The method according to claim 34, further comprising
a step of measuring a trait value for the target trait for each individual or progeny population obtained by crossing a paternal line and a maternal line, and listing the SNPs in the order of the trait value.

6. The method according to claim 34, wherein
the trait value is measured by the target trait for each individual or progeny population obtained by crossing a paternal line and a maternal line, and the SNPs are listed in the order of the trait value.

7. The method according to claim 34, wherein
the SNP of step (1) is selected as a homozygous SNP among polymorphic SNPs between parent lines, having a Minor Allele Frequency (MAF) of 5% or more and missing data of 30% or less.

8. The method according to claim 34, wherein
the target trait-associated SNP marker is selected by repeatedly performing step (1) and step (2) on individuals at both ends obtained by sequentially applying a range of significance probability (p-value) of 5% or less, 10% or less, and 15% or less to both ends of the aligned trait value in step (1).

9. The method according to claim 34, wherein
the target trait-associated SNP marker is selected from a subject.

10. The method of claim 9, wherein
the subject includes one or more selected from the group consisting of a seed, a plant, an animal, a bacterium, and an insect.

11. The method of claim 9, wherein
the subject is a plant.

12. The method of claim 9, wherein
the subject is a seed.

13. The method according to claim 34, wherein
the target trait-associated SNP marker is selected from a sample.

14. The method of claim 13, wherein
the sample includes one or more selected from the group consisting of a seed, a plant, an animal, a bacterium, and an insect.

15. The method of claim 13, wherein
the sample is a plant or a seed.

16. The method according to claim 34, wherein
the target trait has a characteristic including ripening of a pepper fruit.

17. The method according to claim 34, wherein
the target trait has a characteristic including resistance to radish Fusarium wilt.

18. The method according to claim 34, wherein
the target trait has a characteristic including a spicy taste gene (Pun1) of pepper.

19. The method according to claim 34, wherein
the target trait has a characteristic including a resistance gene R1 to Phytophthora pathogen.

20. The method according to claim 34, wherein
the target trait has a characteristic including a resistance gene R8 to Phytophthora pathogen.

21. The method according to claim 34, wherein
the target trait has a characteristic including a resistance gene Rpi-amr1 to Phytophthora pathogen.

22. The method according to claim 34, wherein
the target trait has a characteristic including one or more selected from the group consisting of herbicide resistance, disease resistance, insect or pest resistance, fungal disease resistance, virus resistance, nematode resistance, bacterial disease resistance, fatty acid biosynthesis, starch biosynthesis, increased grain yield, increased oil content, nutritional improvement, increased growth rate, fruit ripening, increased yield, improved stress tolerance, improved environmental or chemical tolerance, change in morphological characteristics, improved digestibility, industrial enzyme production, improved flavor, nitrogen fixation, and a combination thereof.

23. The method according to claim 34, wherein
the method demonstrates superior target trait selection efficiency compared to a GWAS (Genome-Wide Association Study) program and/or QTL mapping.

24. The method according to claim 34, wherein
the method includes
(1) a step of obtaining a single nucleotide polymorphism (SNP) by analyzing genomic sequence information of each individual or progeny population obtained by crossing a male parent and a female parent;
(2) a step of measuring a trait value for a target trait in each individual or progeny population obtained by crossing the male parent and the female parent and listing the SNPs in the order of the trait values;
(3) a step of calculating a score (Sj) of each locus using the following [Formula 1] by applying different weights according to variance based on a genotype of an individual distributed at both ends of the aligned trait values and a genotype of an individual having a median value of the target trait;
$${S}_{j} = {\sum }_{i = 1}^{N} {( {X}_{i} - \overline{X} )}^{2} \cdot {G}_{ij} \qquad [ Formula 1 ]$$
(In [Formula 1], N denotes the total number of samples, Xᵢ denotes the trait value of the i-th sample, X̅ denotes the average of the trait values of all samples, and Gᵢⱼ denotes the sample-specific weighting factor (where Gᵢⱼ=1 if the sample is a homozygous type and the SNP base matches the target trait, Gᵢⱼ=−1 if the sample is a homozygous type and the SNP base does not match the target trait, and Gᵢⱼ=0 if the sample base information is unavailable or the sample is a heterozygous type)); and
(4) a step of selecting a top 10 loci among markers (positions) between 1 and L using [Formula 3] for the score (Sj) of each locus in step (3). [Formula 3]
Top10Positions_{common}=∩_{p∈{5, 10, 20}}SelectTop10({S_{j, p}|j=1, . . . ,L})
(In [Formula 2], L denotes the last marker (position), and P denotes the percentage of samples selected from both ends (upper and lower) in step (1)).

25. The method according to claim 24, wherein
the SNP of step (1) is selected as a homozygous SNP among polymorphic SNPs between parent lines, having a Minor Allele Frequency (MAF) of 5% or more and missing data of 30% or less.

26. The method according to claim 24, wherein
the target trait-associated SNP marker is selected by repeatedly performing step (3) and step (4) on individuals at both ends obtained by sequentially applying a range of significance probability (p-value) of 5% or less, 10% or less, and 15% or less to both ends of the aligned trait value in step (3).

27. A method for producing a plant or seed population, comprising:
determining, detecting, selecting, or isolating a single nucleotide polymorphism (SNP) marker associated with a target trait, comprising:
(1) calculating a score (Sⱼ) for each locus using [Formula 1] by assigning different weights according to variance based on a genotype of an individual distributed at both ends of aligned trait values and a genotype of an individual having a median value of a target trait, for each of at least ten or more reference SNPs; and
$${S}_{j} = {\sum }_{i = 1}^{N} {( {X}_{i} - \overline{X} )}^{2} \cdot {G}_{ij} \qquad [ Formula 1 ]$$
(In [Formula 1], N denotes the total number of samples, Xᵢ denotes the trait value of the i-th sample, X̅ denotes the average of the trait values of all samples, and Gᵢⱼ denotes the sample-specific weighting factor (where Gᵢⱼ=1 if the sample is a homozygous type and the SNP base matches the target trait, Gᵢⱼ=−1 if the sample is a homozygous type and the SNP base does not match the target trait, and Gᵢⱼ=0 if the sample base information is unavailable or the sample is a heterozygous type),
(2) a step of selecting top Q loci among markers (positions) between 1 and L using [Formula 2] for the score (Sj) of each locus in the step (1). [Formula 2]
TopQPositions_{common}=∩_{p∈{5, 10, 20}}SelectTopQ({S_{j, p}|j=1, . . . ,L})
(In [Formula 2], L denotes the last marker (position), P denotes the percentage of samples selected from both ends (upper and lower) in step (1), and Q denotes an integer from 1 to 100;
selecting a plant or seed population; and
crossing or self-fertilizing the selected one or more plants, or plants grown from the selected seeds.

28. The method according to claim 27, wherein
the selected population has a target trait with improved ripening of a pepper fruit.

29. The method according to claim 27, wherein
the selected population has a target trait with improved resistance to radish Fusarium wilt.

30. The method according to claim 27, wherein
the selected population has a target trait with an enhanced spicy taste gene (Pun1) of pepper.

31. The method according to claim 27, wherein
the selected population has a target trait with an enhanced resistance gene R1 against Phytophthora pathogen.

32. The method according to claim 27, wherein
the selected population has a target trait with an enhanced resistance gene R8 against Phytophthora pathogen.

33. The method according to claim 27, wherein
the selected population has a target trait with an enhanced resistance gene Rpi-amr1 against Phytophthora pathogen.

34. A method for introducing or increasing expression of a target trait-related SNP in a subject, comprising:
determining, detecting, selecting, or isolating a single nucleotide polymorphism (SNP) marker associated with a target trait, comprising:
(1) calculating a score (Si) for each locus using [Formula 1] by assigning different weights according to variance based on a genotype of an individual distributed at both ends of aligned trait values and a genotype of an individual having a median value of a target trait, for each of at least ten or more reference SNPs; and
$${S}_{j} = {\sum }_{i = 1}^{N} {( {X}_{i} - \overline{X} )}^{2} \cdot {G}_{ij} \qquad [ Formula 1 ]$$
(In [Formula 1], N denotes the total number of samples, Xᵢ denotes the trait value of the i-th sample, X̅ denotes the average of the trait values of all samples, and Gᵢⱼ denotes the sample-specific weighting factor (where Gᵢⱼ=1 if the sample is a homozygous type and the SNP base matches the target trait, Gᵢⱼ=−1 if the sample is a homozygous type and the SNP base does not match the target trait, and Gᵢⱼ=0 if the sample base information is unavailable or the sample is a heterozygous type),
(2) a step of selecting top Q loci among markers (positions) between 1 and L using [Formula 2] for the score (Sj) of each locus in the step (1). [Formula 2]
TopQPositions_{common}=∩_{p∈{5, 10, 20}}SelectTopQ({S_{j, p}|j=1, . . . ,L})
(In [Formula 2], L denotes the last marker (position), P denotes the percentage of samples selected from both ends (upper and lower) in step (1), and Q denotes an integer from 1 to 100; and
expressing or increasing the expression of the target trait-related SNP in the subject.

35. A method for producing a plant having a target trait, comprising:
determining, detecting, selecting, or isolating a single nucleotide polymorphism (SNP) marker associated with a target trait, comprising:
(1) calculating a score (Si) for each locus using [Formula 1] by assigning different weights according to variance based on a genotype of an individual distributed at both ends of aligned trait values and a genotype of an individual having a median value of a target trait, for each of at least ten or more reference SNPs; and
$${S}_{j} = {\sum }_{i = 1}^{N} {( {X}_{i} - \overline{X} )}^{2} \cdot {G}_{ij} \qquad [ Formula 1 ]$$
(In [Formula 1], N denotes the total number of samples, Xᵢ denotes the trait value of the i-th sample, X̅ denotes the average of the trait values of all samples, and Gᵢⱼ denotes the sample-specific weighting factor (where Gᵢⱼ=1 if the sample is a homozygous type and the SNP base matches the target trait, Gᵢⱼ=−1 if the sample is a homozygous type and the SNP base does not match the target trait, and Gᵢⱼ=0 if the sample base information is unavailable or the sample is a heterozygous type),
(2) a step of selecting top Q loci among markers (positions) between 1 and L using [Formula 2] for the score (Sj) of each locus in the step (1). [Formula 2]
TopQPositions_{common}=∩_{p∈{5, 10, 20}}SelectTopQ({S_{j, p}|j=1, . . . ,L})
(In [Formula 2], L denotes the last marker (position), P denotes the percentage of samples selected from both ends (upper and lower) in step (1), and Q denotes an integer from 1 to 100; and
expressing or increasing the expression of the target trait-related SNP in the subject.

36. The method according to claim 34, wherein
the subject is a plant, and the method further includes crossing the plant with another plant of the same species to produce a progeny plant having the target trait.

37. A plant having a target trait produced by the method according to claim 34.
